SEQUENCE LISTING (25 sequences)

SEQ ID NO: 1 - DNA, 23 bp, Homo sapiens
aaattctcagaatttctgaaagg

SEQ ID NO: 2 - DNA, 22 bp, Homo sapiens
agtgcttaacaagaccatacta

SEQ ID NO: 3 - DNA, 22 bp, Homo sapiens
agtgcttaacaagaccatacta

SEQ ID NO: 4 - DNA, 24 bp, Homo sapiens
aacagacctggaatttgaaactaa

SEQ ID NO: 5 - DNA, 20 bp, Homo sapiens
attactggctcttcctgagc

SEQ ID NO: 6 - DNA, 20 bp, Homo sapiens
ttctgggctcctggaaccaa

SEQ ID NO: 7 - DNA, 20 bp, Homo sapiens
tctagaaacagttgcctggc

SEQ ID NO: 8 - DNA, 20 bp, Homo sapiens
ctaccagcgtgccaccaggt

SEQ ID NO: 9 - DNA, 16 bp, Homo sapiens
  misc_feature (13)..(13): n is inosine
ctggacaggcgangaa

SEQ ID NO: 10 - DNA, 16 bp, Homo sapiens
  misc_feature (13)..(13): n is inosine
ctggacaggcaangaa

SEQ ID NO: 11 - DNA, 18 bp, Homo sapiens
actctcagcaagcctcaa

SEQ ID NO: 12 - DNA, 18 bp, Homo sapiens
  misc_feature (13)..(13): n is inosine
actctcagcgagnctcaa

SEQ ID NO: 13 - DNA, 51 bp, Artificial Sequence
  T7 Phage Polymerase Promoter
aattctaatacgactcactatagggagaaaattctcagaatttctgaaagg

SEQ ID NO: 14 - DNA, 51 bp, Artificial Sequence
  T7 Phage Polymerase Promoter
aattctaatacgactcactatagggagaaggattactggctcttcctgagc

SEQ ID NO: 15 - DNA, 20 bp, Homo sapiens
agctgcccatgaatagcact

SEQ ID NO: 16 - DNA, 21 bp, Homo sapiens
ctggaaccaatcccgtgaaag

SEQ ID NO: 17 - DNA, 18 bp, Homo sapiens
actctcagcaagcctcaa

SEQ ID NO: 18 - DNA, 15 bp, Homo sapiens
tctcagcgggcctca

SEQ ID NO: 19 - DNA, 44 bp, Artificial Sequence
  T7 Phage Polymerase Promoter
aattctaatacgactcactataggagctgcccatgaatagcact

SEQ ID NO: 20 - DNA, 28 bp, Homo sapiens
  misc_feature (19)..(19): n is inosine
cgatcgctggacaggcgangaacgatcg

SEQ ID NO: 21 - DNA, 28 bp, Homo sapiens
  misc_feature (19)..(19): n is inosine
cgatcgctggacaggcaangaacgatcg

SEQ ID NO: 22 - DNA, 30 bp, Homo sapiens
cgatcgactctcagcaagcctcaacgatcg

SEQ ID NO: 23 - DNA, 30 bp, Homo sapiens
  misc_feature (19)..(19): n is inosine
cggtcgactctcagcgagnctcaacgaccg

SEQ ID NO: 24 - DNA, 30 bp, Homo sapiens; identical to SEQ ID NO: 22

SEQ ID NO: 25 - DNA, 27 bp, Homo sapiens
cgatcgtctcagcgggcctcacgatcg

(19) United States
(12) Patent Application Publication
(10) Pub. No.: US 2007/0160996 A1
(43) Pub. Date: Jul. 12, 2007

(54) Thrombosis diagnosis/prognosis method

(75) Inventors: Philippe Leissner, Lyon (FR); Aurelie Brion, Vaugneray (FR); Cecile Berard, Lyon (FR)

Correspondence Address: OLIFF & BERRIDGE, PLC, P.O. BOX 19928, ALEXANDRIA, VA, 22320, US

(73) Assignee: BIOMERIEUX, Marcy L'Etoile (FR)

(21) Appl. No.: 10/585,923

(22) PCT Filed: Feb. 10, 2005
(86) PCT No.: PCT/FR05/50083

§ 371 Date: Jul. 20, 2006

(30) Foreign Application Priority Data
Feb. 12, 2004 (FR) 0450257

(51) Int. Cl. C12Q 1/68; C07H 21/04; C12P 19/34

(52) U.S. Cl. 435/6; 435/91.2; 536/24.3

(57) ABSTRACT

The invention relates to an in vitro method for the diagnosis/prognosis of thrombosis, comprising the following steps: A—the nucleic material is extracted from a biological sample, B—at least one pair of amplification primers is used to obtain amplicons of at least one target sequence of the nucleic material, C—at least one detection probe is used to detect the presence of said amplicons, characterized in that, in step B, said pair of primers comprises at least one amplification primer comprising at least 10 nucleotide units of a nucleotide sequence chosen from SEQ ID Nos. 1; 3 to 8, 15 and 16.

The present invention relates to an in vitro method for the diagnosis/prognosis of thrombosis. The invention also relates to amplification primers and hybridization probes which can be used in this method, and also to a kit for the diagnosis/prognosis of thrombosis.

Coagulation is an essential homeostatic system which must be correctly regulated in order to avoid hemorrhages and thromboses.

This coagulation involves a cascade of activations of inactive circulating precursors which, through the loss of one end of their protein chain, give activated factors. Among these coagulation factors, mention may in particular be made of factor II and factor V. Factor II, also called prothrombin, is a proenzyme synthesized by the liver. It is activated to thrombin, the active form, by factors X and V activated in the presence of phospholipids. When it is activated, it is responsible for the proteolysis of fibrinogen to fibrin.

Among the known impairments of homeostasis, mention may in particular be made of arterial thromboses, which manifest themselves in the form of myocardial infarction, attacks, and through peripheral arterial diseases, and also venous embolic thromboses which are major causes of morbidity and mortality. Mutations in the genes encoding factors II and V are thought to be risk factors for these impairments. Thus, in 1996, the study of the coding and adjacent regions of the gene encoding factor II, in a family with various individuals suffering from venous thrombosis, made it possible to demonstrate a mutation at position 20210 of the gene (hereinafter called 20210 mutation). This mutation leads to a 25% increase in the activity of thrombin in the plasma and is considered to be a risk factor for venous thromboses.

The mutation of nucleotide 1691 of the gene encoding factor V, called factor V Leiden mutation, is also a risk factor. This mutation increases the risk of venous thrombosis by 4- to 8-fold in heterozygote individuals and by 50- to 100-fold for homozygotes.

Early detection of factor II 20210 mutations and the factor V Leiden mutation is therefore essential in order to prevent as early as possible the risks of venous thrombosis. Furthermore, many studies show that the factor II 20210 mutation and the factor V Leiden mutation are often coinherited, and the presence of the two mutations induces an increase in the risk of venous thrombosis. It is therefore also important to study these two mutations simultaneously.

U.S. Pat. No. 6,558,913 presents a method for detecting the presence of a genetic risk of thrombosis, based on the detection of the factor V Leiden mutation. Mention may also be made of U.S. Pat. No. 6,043,035, which presents a method for detecting the presence of a genetic risk of thrombosis, based on the detection of the factor II 20210 mutation. These methods comprise in particular a step consisting in amplifying the region of the gene in which the mutation is located, using amplification primers, followed by a step consisting in detecting the mutation using a detection probe specific for the mutation. These methods do not, however, make it possible to simultaneously detect the factor V Leiden mutation and the factor II 20210 mutation, which nevertheless greatly increases the risk of thrombosis in a patient. The early detection of this double mutation, which can be carried out sequentially or separately, makes it possible to warn the patient and to propose to the latter a suitable treatment. Furthermore, these methods can be optimized through the choice of the amplification primers used in the amplification step, in order to improve the detection step, all the more so when the amplification step is carried out simultaneously with the detection step.

In this respect, the present invention proposes to improve the prior art by providing a novel method for evaluating a patient's risk of thrombosis. This method in particular uses, during the amplification step, novel amplification primers that are perfectly suitable for the detection of a mutation that increases the risk of thrombosis. These novel amplification primers can also be used in the same amplification reaction, which makes it possible in particular to determine, by means of a single reaction, the presence of factor II 20210 mutations and the factor V Leiden mutation.

In this respect, the invention relates to an in vitro method for the diagnosis/prognosis of thrombosis, comprising the following steps:
A—the nucleic material is extracted from a biological sample,
B—at least one pair of amplification primers is used to obtain amplicons of at least one target sequence of the nucleic material,
C—at least one detection probe is used to detect the presence of said amplicons,
   characterized in that, in step B, said pair of primers comprises at least one amplification primer comprising at least 10 nucleotide units of a nucleotide sequence chosen from SEQ ID Nos. 1; 3 to 8, 15 and 16.

For the purpose of the present invention, the term “diagnosis/prognosis of thrombosis” is intended to mean the establishment of a thromophilia genetic risk profile.

For the purposes of the present invention, the term “biological sample” is intended to mean any sample that may contain a nucleic material as defined hereinafter. This biological sample can be taken from a patient and can in particular be a tissue, blood, serum, salival or circulating cell sample from the patient. This biological sample is obtained by any type of sampling method known to those skilled in the art.

For the purpose of the present invention, the nucleic material comprises a sequence of nucleic acids such as a sequence of deoxyribonucleic acids (DNA) or ribonucleic acids (RNA). According to a preferred embodiment of the invention, the nucleic material comprises a sequence of deoxyribonucleic acids. According to a preferred embodiment of the invention, the nucleic material is extracted from a biological sample taken from a patient. The term “nucleotide sequence” (or nucleic acid sequence or nucleotide fragment or oligonucleotide or polynucleotide) is intended to mean a series of nucleotide units assembled together via phosphoric ester bonds, characterized by the informational sequence of the natural nucleic acids capable of hybridizing to another nucleic acid sequence, it being possible for the series to contain monomers of different structures and to be obtained from a natural nucleic acid molecule and/or by genetic recombination and/or by chemical synthesis. The term “nucleotide unit” is intended to mean a derivative of a monomer which can be a natural nucleotide of nucleic acid in which the consecutive elements are a sugar, a phosphate group and a nitrogenous base; in DNA, the sugar is 2-deoxyribose, in RNA, the sugar is ribose; depending on whether it is a question of DNA or RNA, the nitrogenous base is chosen from adenine, guanine, uracil, cytosine and thymine; or else the monomer is a nucleotide modified in at least one of the three constitutive elements; by way of example, the modification can occur either at the level of the bases, with modified bases such as inosine, methyl-5-deoxycytidine, deoxyuridine, dimethylamino-5-deoxyuridine, diamino-2,6-purine, bromo-5-deoxyuridine or any other base capable of hybridization, or at the level of the sugar, for example the replacement of at least one deoxyribose with a polyamide (P. E. Nielsen et al, Science, 254, 1497-1500 (1991)), or else at the level of the phosphate group, for example the replacement thereof with esters chosen in particular from diphosphates, alkylphosphonates, arylphosphonates and phosphorothioates. This nucleic material comprises at least one target sequence.

The term “target sequence” is intended to mean a sequence in which the series of nucleotide units is specific for a target gene, such as preferably the gene encoding factor II or the gene encoding factor V. For the purpose of the present invention, the target sequence comprises a mutation which increases the risk of thrombosis, such as in particular the factor V Leiden mutation or the factor II 20210 mutation. In the subsequent disclosure, reference will be made to a target sequence, whether it is single-stranded or double-stranded.

During step A, the nucleic material is extracted from a biological sample by any protocol known to those skilled in the art. By way of indication, the nucleic acid extraction can be carried out by means of a step consisting of lysis of the cells present in the biological sample, in order to release the nucleic acids contained in the protein and/or lipid envelopes of the cells (such as cell debris which disturbs the subsequent reactions). By way of example, use may be made of the lysis methods as described in patent application WO 00/05338 regarding mixed magnetic and mechanical lysis, patent application WO 99/53304 regarding electrical lysis, and patent application WO 99/15321 regarding mechanical lysis.

Those skilled in the art may use other well known lysis methods, such as heat shock or osmotic shock or chemical lysis with chaotropic agents such as guanidium salts (U.S. Pat. No. 5,234,809). This lysis step can also be followed by a purification step, allowing separation between the nucleic acids and the other cellular constituents released in the lysis step. This step generally makes it possible to concentrate the nucleic acids, and can be adapted to the purification of DNA or of RNA. By way of example, use may be made of magnetic particles optionally coated with oligonucleotides, by absorption or covalence (in this respect, see patents U.S. Pat. No. 4,672,040 and U.S. Pat. No. 5,750,338), and the nucleic acids which are attached to these magnetic particles can thus be purified by means of a washing step. This nucleic acid purification step is particularly advantageous if it is desired to subsequently amplify said nucleic acids. A particularly advantageous embodiment of these magnetic particles is described in patent applications WO 97/45202 and WO 99/35500. Another advantageous example of a method for purifying nucleic acids is the use of silica in the form of a column, or in the form of inert particles (Boom R. et al., J. Clin. Microbiol., 1990, n^{o}28(3), p. 495-503) or magnetic particles (Merck: MagPrep® Silica, Promega: MagneSil™ Paramagnetic particles). Other very widely used methods are based on ion exchange resins in a column or in a paramagnetic particulate formate (Whatman: DEAE-Magarose) (Levison P R et al., J. Chromatography, 1998, p. 337-344). Another method which is very relevant but not exclusive for the invention is that of adsorption onto a support of metal oxide (the company Xtrana: Xtra-Bind™ matrix).

When it is desired to specifically extract the DNA from a biological sample, an extraction can in particular be carried out with phenol, chloroform and alcohol in order to remove the proteins and precipitate the DNA with the alcohol. The DNA can then be pelleted by centrifugation, washed and resuspended.

During step B, at least one pair of amplification primers is used to obtain amplicons of at least one target sequence of the nucleic material.

For the purpose of the present invention, the term “amplification primer” is intended to mean a nucleic sequence comprising from 10 to 100 nucleotide units, preferably from 15 to 25 nucleotide units. This amplification primer comprises at least 10, preferably 15, and even more preferably 20, nucleotide units of a sequence chosen from SEQ ID Nos. 1; 3 to 8

For the purpose of the present invention, an amplification primer comprising at least 10, preferably 15, and even more preferably 20, nucleotide units of
a sequence homologous to SEQ ID Nos. 1; 3 to SEQ ID Nos. 8; 15 and 16, i.e.
   the sequence complementary to SEQ ID Nos. 1; 3 to 8; 15 and 16,
   a sequence exhibiting sufficient homology to hybridize to SEQ ID Nos. 1; 3 to SEQ ID Nos. 8; 15 and 16 or to the sequence complementary to SEQ ID Nos. 1; 3 to SEQ ID Nos. 8; 15 and 16,
a sequence comprising a sequence of SEQ ID Nos. 1; 3 to SEQ ID Nos. 8; 15 and 16 (or a sequence homologous to SEQ ID Nos. 1; 3 to SEQ ID Nos. 8; 15 and 16 as defined above) in which the uracil bases are substituted with thymine bases,
   and which would have the same function as the amplification primer according to the invention, i.e. that amplifying all or part of the gene coding factor V (SEQ ID Nos. 1; 3 to 4) that may contain the Leiden mutation or all or part of the gene encoding factor II (SEQ ID Nos. 5 to 8; 15 and 16), that may contain the 20210 mutation, is considered to be equivalent to the amplification primer according to the invention.

A pair of amplification primers makes it possible to initiate an enzymatic polymerization, such as in particular an enzymatic amplification reaction.

The term “enzymatic amplification reaction” is intended to mean a process generating multiple copies (or amplicons) of a nucleic sequence through the action of at least one enzyme. For the purpose of the present invention, the term “amplicons” is intended to mean the copies of the target sequence obtained during an enzymatic amplification reaction. Such amplification reactions are well known to those skilled in the art, and mention may in particular be made of PCR (Polymerase Chain Reaction), as described in patents U.S. Pat. No. 4,683,195, U.S. Pat. No. 4,683,202 and U.S. Pat. No. 4,800,159; LCR (Ligase Chain Reaction), disclosed, for example, in patent application EP-A-0 201 184; RCR (Repair Chain Reaction), described in patent application WO-A-90/01069; 3SR (Self Sustained Sequence Replication) with patent application WO-A-90/06995; NASBA (Nucleic Acid Sequence-Based Amplification) with patent application WO-A-91/02818, or else TMA (Transcription Mediated Amplification) with patent U.S. Pat. No. 5,399,491.

In general, these enzymatic amplification reactions generally use a succession of cycles comprising the following steps:
denaturation of the target sequence if the latter is double-stranded, in order to obtain two target strands which are complementary,
hybridization of each of the target strands, obtained during the preceding denaturation step, with at least one amplification primer,
formation, from the amplification primers, of the strands complementary to the strands to which they are hybridized, in the presence of a polymerase enzyme and of nucleoside triphosphates (ribonucleoside triphosphate and/or deoxyribonucleoside triphosphate according to techniques),
   this cycle being repeated a given number of times so as to obtain the target sequence in a proportion sufficient to allow the detection thereof.

The term “hybridization” is intended to mean the process during which, under appropriate conditions, two nucleic sequences, such as in particular an amplification primer and a target sequence or a hybridization probe and a target sequence, bind to one another with stable and specific hydrogen bonds so as to form a double strand. These hydrogen bonds form between the complementary bases adenine (A) and thymine (T) (or uracil (U)) (reference is made to an A-T bond) or between the complementary bases guanine (G) and cytosine (C) (reference is made to a G-C bond). The hybridization of two nucleic sequences can be complete (reference is then made to complementary sequences), i.e. the double strand obtained during this hybridization comprises only A-T bonds and C-G bonds. This hybridization may be partial (reference is then made to sufficiently complementary sequences), i.e. the double strand obtained comprises A-T bonds and C-G bonds that make it possible to form the double strand, but also bases not bonded to a complementary base. The hybridization between two complementary sequences or sufficiently complementary sequences depends on the operating conditions which are used, and in particular on the stringency. The stringency is defined in particular as a function of the base composition of the two nucleic sequences, and also by virtue of the degree of mismatching between these two nucleic sequences. The stringency can also depend on the reaction parameters, such as the concentration and the type of ionic species present in the hybridization solution, the nature and the concentration of denaturing agents and/or the hybridization temperature. All these data are well known and the appropriate conditions can be determined by those skilled in the art.

More specifically, NASBA is a technology consisting of isothermal amplification of the nucleic acid, based on the joint action of three enzymes (AMV reverse transcriptase, Rnase-H and T7 RNA polymerase). Combined with amplification primers specific for a target sequence, it amplifies the RNA targets more than one billion times in 90 minutes. The amplification reaction is carried out at 41° C. and gives single-stranded RNA molecules as the final product. NASBA requires a pair of primers, at least one of which comprises a promoter for the initiation of transcription by a T7 bacteriophage polymerase.

During step C, at least one detection probe is used to detect the presence of said amplicons. Preferably, this detection probe makes it possible to detect not only the presence of said amplicons, but also makes it possible to detect the presence of a given mutation that may be included in the amplicon.

This detection step can be carried out by any of the protocols known to those skilled in the art concerning the detection of nucleic acids.

For the purpose of the present invention, the term “hybridization probe” is intended to mean a nucleic sequence having a hybridization specificity under given conditions for forming a hybridization complex with a target nucleic sequence. The hybridization probe can comprise a marker for its detection. Reference is then made to detection probes. The term “detection” is intended to mean either a direct detection by means of a physical method, or an indirect detection by means of a method of detection using a label. Many detection methods exist for the detection of nucleic acids [see, for example, Kricka et al., Clinical Chemistry, 1999, No. 45(4), p. 453-458 or Keller G. H. et al., DNA Probes, 2nd Ed., Stockton Press, 1993, sections 5 and 6, p. 173-249]. The term “label” is intended to mean a tracer capable of engendering a signal that can be detected. A nonlimiting list of tracers includes enzymes which produce a signal that is detectable, for example by colorimetry, fluorescence or luminescence, such as horseradish peroxidase, alkaline phosphatase, beta-galactosidase or glucose-6-phosphate dehydrogenase; chromophores such as fluorescent, luminescent or dye compounds; electron-dense groups that are detectable by electron microscopy or through their electrical properties such as conductivity, by amperometry or voltametry methods, or by impedance measurements; groups that are detectable by optical methods such as diffraction, surface plasmon resonance or contact angle variation, or by physical methods such as atomic force spectroscopy, tunnel effect, etc.; radioactive molecules such as ³²P, ³⁵S or ¹²⁵I.

The detection probe can in particular be a “molecular beacon” detection probe as described by Tyagi & Kramer (Nature biotech, 1996, 14:303-308). These “molecular beacons” become fluorescent during hybridization. They have a stem-loop structure and contain a fluorophore and an inhibitor group or “quencher”. The binding of the specific loop sequence with its complementary target nucleic acid sequence causes unfolding of the stem and the emission of a fluorescent signal during excitation at the appropriate wavelength.

According to a preferred embodiment of the invention, the detection probe comprises a fluorophore and a quencher. According to an even more preferred embodiment of the invention, the hybridization probe comprises a FAM (6-carboxyfluorescene) or ROX (6-carboxy-X-rhodamine) fluorophore at its 5′ end and a quencher (Dabsyl) at its 3′ end. In the subsequent disclosure, such a hybridization probe is called a “molecular beacon”. According to a preferred embodiment of the invention, during step C), the presence or absence of the Leiden factor V mutation is detected. According to a preferred embodiment of the invention, during step C), the presence or absence of the factor II 20210 mutation is detected. According to an even more preferred embodiment, the presence or absence of the factor V Leiden mutation and of the factor II 20210 mutation are simultaneously detected.

According to a preferred embodiment of the invention, during step C), said detection probe comprises at least 10, preferably 15, and even more preferably 20, nucleotide units of a nucleotide sequence chosen from SEQ ID Nos. 9 to 12; 17 and 18. Thus, the use of a detection probe comprising SEQ ID No. 9 makes it possible to diagnose the presence of the factor V Leiden mutation, whereas the use of a detection probe comprising SEQ ID No. 10 makes it possible to diagnose the absence of the factor V Leiden mutation.

Similarly, the use of a detection probe comprising SEQ ID No. 11 makes it possible to detect the presence of the factor II 20210 mutation, whereas the use of a detection probe comprising SEQ ID No. 12 makes it possible to detect the absence of the factor II 20210 mutation. Furthermore, the use of a detection probe comprising SEQ ID No. 17 makes it possible to detect the presence of the factor II 20210 mutation, whereas the use of a detection probe comprising SEQ ID No. 18 makes it possible to detect the absence of the factor II 20210 mutation.

According to a preferred embodiment of the invention, during step B, said pair of primers is chosen from the following pairs of primers:
a first amplification primer comprising at least 10, preferably 15, and even more preferably 20, nucleotide units of the nucleotide sequence SEQ ID No. 1 and a second amplification primer comprising at least 10, preferably 15, and even more preferably 20, nucleotide units of the nucleotide sequence SEQ ID No. 2; by way of indication, when the first primer has the sequence SEQ ID No. 1, and the second primer has the sequence SEQ ID No. 2, an amplicon, specific for the gene encoding factor V, 159 base pairs in size, which corresponds to the sequence 36568-36726 on a sequence of the reference gene encoding the factor V (NT 004668), is obtained. This amplicon can contain the mutated allele responsible for the Leiden mutation or the wild-type allele, the allele being characterized during step C;
a first amplification primer comprising at least 10, preferably 15, and even more preferably 20, nucleotide units of the nucleotide sequence SEQ ID No. 3 and a second amplification primer comprising at least 10, preferably 15, and even more preferably 20 nucleotide units of the nucleotide sequence SEQ ID No. 4; by way of indication, when the first primer has the sequence SEQ ID No. 3, and the second primer has the sequence SEQ ID No. 4, an amplicon, specific for the gene encoding factor V, 374 base pairs in size, which corresponds to the sequence 36568-36941 on the sequence of the reference gene encoding factor V (NT_{—}004668), is obtained. This amplicon can contain the mutated allele responsible for the Leiden mutation or the wild-type allele, the allele being characterized during step C;
a first amplification primer comprising at least 10, preferably 15, and even more preferably 20, nucleotide units of the nucleotide sequence SEQ ID No. 5 and a second amplification primer comprising at least 10, preferably 15, and even more preferably 20, nucleotide units of the nucleotide sequence SEQ ID No. 6; by way of indication, when the first primer has the sequence SEQ ID No. 5, and the second primer has the sequence SEQ ID No. 6, an amplicon, specific for the gene encoding factor II, 145 base pairs in size, which corresponds to the sequence 21455-21599 on the sequence of the reference gene encoding factor II (AF478696), is obtained. This amplicon can contain the mutated allele responsible for the factor II 20210 mutation or the wild-type allele, the allele being characterized during step C;
a first amplification primer comprising at least 10, preferably 15, and even more preferably 20, nucleotide units of the nucleotide sequence SEQ ID No. 7, and a second amplification primer comprising at least 10, preferably 15, and even more preferably 20, nucleotide units of the nucleotide sequence SEQ ID No. 8; by way of indication, when the first primer has the sequence SEQ ID No. 7, and the second primer has the sequence SEQ ID No. 8, an amplicon, specific for the gene encoding factor II, 434 base pairs in size, which corresponds to the sequence 21217-21650 on the sequence of the reference gene encoding factor II (AF478696), is obtained. This amplicon can contain the mutated allele responsible for the factor II 20210 mutation or the wild-type allele, the allele being characterized during step C;
a first amplification primer comprising at least 10, preferably 15, and even more preferably 20, nucleotide units of the nucleotide sequence SEQ ID No. 15 and a second amplification primer comprising at least 10, preferably 15, and even more preferably 20, nucleotide units of the nucleotide sequence SEQ ID No. 16; by way of indication, when the first primer has the sequence SEQ ID No. 15, and the second primer has the sequence SEQ ID No. 16, an amplicon, specific for the gene encoding factor II, 108 base pairs in size, which corresponds to the sequence 21465-21573 on the sequence of the reference gene encoding factor II (AF478696), is obtained. This amplicon can contain the mutated allele responsible for the factor II 20210 mutation, or the wild-type allele, the allele being characterized during step C.

According to a preferred embodiment of the invention, said pair of primers comprises at least one amplification primer comprising a promoter allowing the initiation of transcription by a T7 bacteriophage polymerase. Preferably, this first amplification primer comprises a sequence chosen from the sequences SEQ ID Nos. 13 to 14.

According to a preferred embodiment of the invention, step B and step C are carried out simultaneously. This can be implemented in particular by means of an NASBA amplification reaction with real-time detection. The real-time detection of the amplicons can be carried out using a Nuclisens EasyQ® reader (bioMérieux BV, the Netherlands) and “molecular beacon” detection probes, as defined above. In general, the DNA amplification by NASBA can be carried out in the following way: the DNA is denatured at 95° C. so as to allow the binding of a “first primer”, comprising a T7 polymerase promoter (a single primer is present at this stage). At 41° C., the reverse transcriptase enzyme (AMV-RT) is added and allows primer extension. A “second primer” is then added and a further denaturation is performed. An alternative is carried out by simultaneously adding the two primers, so as to carry out just one denaturation step. After this initiation phase, the NASBA enters into a cyclic phase: the “second primer” hybridizes to a new RNA molecule formed; the AMV-RT extends this primer; the RNase H degrades the RNA of the RNA/cDNA hybrids; a “first primer” binds to the cDNA obtained; the AMV-RT synthesizes, from the latter, a new complementary strand and then, from this strand, extends the T7 part of the “first primer”; thus, the T7 RNA polymerase has new templates with a double T7 tail for generating the RNAs. When the “molecular beacon” is in solution, the quencher, in the vicinity of the flourophor, inhibits the fluorescence thereof. In the presence of the target nucleic acid, the beacon will bind to the sequence complementary to that of a loop. It will therefore change conformation and open up, moving the fluorophore away from the quencher, and thus allowing the emission of fluorescence. During the amplification, numerous molecules will be synthesized, allowing as many “molecular beacons” to bind. The fluorescence will therefore increase during the reaction. An appropriate device, such as the EasyQ Nuclisens Analyzer, makes it possible to record the fluorescence over time.

The present invention also relates to an amplification primer comprising at least 10, preferably 15, and even more preferably 20, nucleotide units of a nucleotide sequence chosen from SEQ ID Nos. 1; 3 to 8; 15 and 16.

According to a preferred embodiment of the invention, the amplification primer comprises a promoter allowing the initiation of transcription by a T7 bacteriophage polymerase. Preferably, such an amplification primer comprises a sequence chosen from the sequences SEQ ID Nos. 13 to 14.

The invention also relates to a pair of amplification primers chosen from the following pairs of primers:
a first amplification primer comprising at least 10, preferably 15, and even more preferably 20, nucleotide units of the nucleotide sequence SEQ ID No. 1 and a second amplification primer comprising at least 10, preferably 15, and even more preferably 20, nucleotide units of the nucleotide sequence SEQ ID No. 2; by way of indication, when the first primer has the sequence SEQ ID No. 1, and the second primer has the sequence SEQ ID No. 2, an amplicon, specific for the gene encoding factor V, 159 base pairs in size, which corresponds to the sequence 36568-36726 on a sequence of the reference gene encoding the factor V (NT 004668), is obtained;
a first amplification primer comprising at least 10, preferably 15, and even more preferably 20, nucleotide units of the nucleotide sequence SEQ ID No. 3 and a second amplification primer comprising at least 10, preferably 15, and even more preferably 20 nucleotide units of the nucleotide sequence SEQ ID No. 4; by way of indication, when the first primer has the sequence SEQ ID No. 3, and the second primer has the sequence SEQ ID No. 4, an amplicon, specific for the gene encoding factor V, 374 base pairs in size, which corresponds to the sequence 36568-36941 on the sequence of the reference gene encoding factor V (NT_{—}004668), is obtained;
a first amplification primer comprising at least 10, preferably 15, and even more preferably 20, nucleotide units of the nucleotide sequence SEQ ID No. 5 and a second amplification primer comprising at least 10, preferably 15, and even more preferably 20, nucleotide units of the nucleotide sequence SEQ ID No. 6; by way of indication, when the first primer has the sequence SEQ ID No. 5, and the second primer has the sequence SEQ ID No. 6, an amplicon, specific for the gene encoding factor II, 145 base pairs in size, which corresponds to the sequence 21455-21599 on the sequence of the reference gene encoding factor II (AF478696), is obtained;
a first amplification primer comprising at least 10, preferably 15, and even more preferably 20, nucleotide units of the nucleotide sequence SEQ ID No. 7, and a second amplification primer comprising at least 10, preferably 15, and even more preferably 20, nucleotide units of the nucleotide sequence SEQ ID No. 8; by way of indication, when the first primer has the sequence SEQ ID No. 7, and the second primer has the sequence SEQ ID No. 8, an amplicon, specific for the gene encoding factor II, 434 base pairs in size, which corresponds to the sequence 21217-21650 on the sequence encoding the reference gene encoding factor II (AF478696), is obtained;
a first amplification primer comprising at least 10, preferably 15, and even more preferably 20, nucleotide units of the nucleotide sequence SEQ ID No. 15 and a second amplification primer comprising at least 10, preferably 15, and even more preferably 20, nucleotide units of the nucleotide sequence SEQ ID No. 16; by way of indication, when the first primer has the sequence SEQ ID No. 15, and the second primer has the sequence SEQ ID No. 16, an amplicon, specific for the gene encoding factor II, 108 base pairs in size, which corresponds to the sequence 21465-21573 on the sequence of the reference gene encoding factor II (AF478696), is obtained.

According to a preferred embodiment of the invention, said first primer comprises a promoter allowing the initiation of transcription by a T7 bacteriophage polymerase. Preferably, this first amplification primer comprises a sequence chosen from the sequences SEQ ID Nos. 13 to 14.

The invention also relates to the use of at least one amplification primer as defined above, and/or of a pair of primers as defined above, in a NASBA amplification reaction. The invention also relates to the use of at least one primer as defined above and/or of at least one pair of primers as defined above, for the diagnosis/prognosis of thrombosis. Finally, the invention relates to a kit for the diagnosis/prognosis of venous thrombosis, comprising at least one primer as defined above and/or at least one pair of primers as defined above.

The following figures are given by way of illustration and are no way limiting in nature. They will make it possible to understand the invention more clearly.

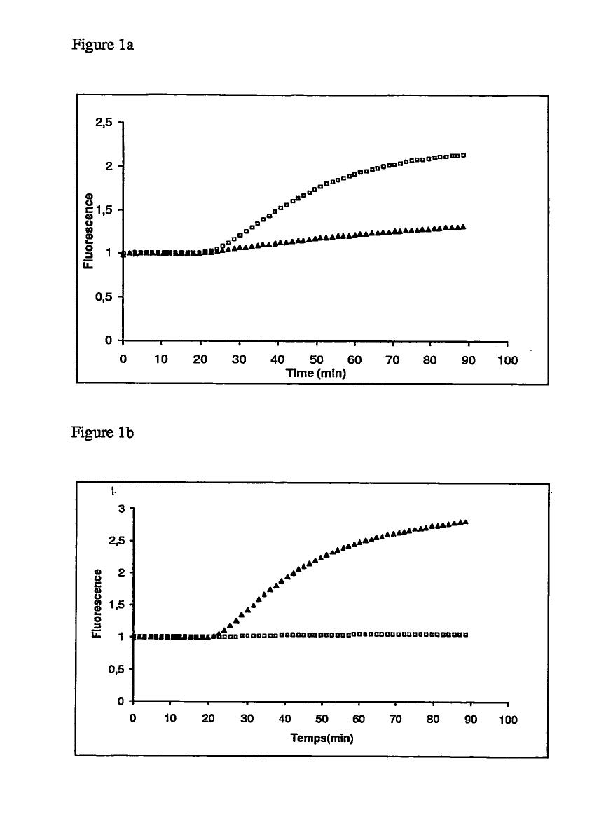

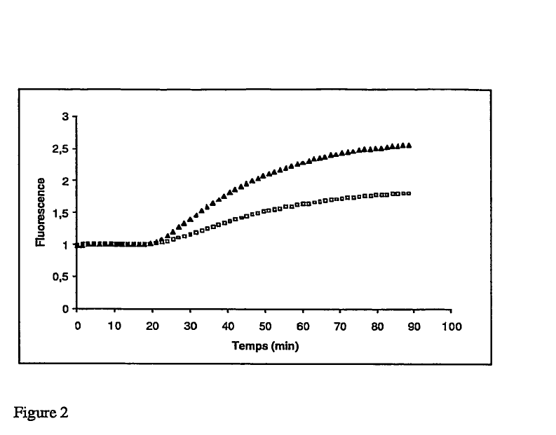

FIGS. 1 and 2 represent the genotyping of various cell lines for the factor V mutation +1691 G/A, by virtue of the simultaneous presence of the molecular beacons of SEQ ID No. 10 and SEQ ID No. 9 in the reaction mixture.

Thus, FIG. 1a represents the genotyping of the GM16000C cell line homozygous for the factor V wild-type allele (+1691-G), while FIG. 1b represents the genotyping of the GM14899 cell line homozygous for the factor V mutated allele (+1691-A). The white squares represent the detection of the fluorescence of the “molecular beacons” of SEQ ID No. 9, making it possible to reveal the factor V wild-type allele, while the black triangles represent the detection of the fluorescence of the “molecular beacons” of SEQ ID No. 10, allowing the detection of the mutated allele (Leiden mutation).

FIG. 2 represents the genotyping of the GM16028B cell line heterozygous for the factor V +1691 mutation. The white squares represent the detection of the fluorescence of the “molecular beacons” of SEQ ID No. 9, making it possible to reveal the factor V wild-type allele, while the black triangles represent the detection of the fluorescence of the “molecular beacons” of SEQ ID No. 10, allowing the detection of the mutated allele (Leiden mutation).

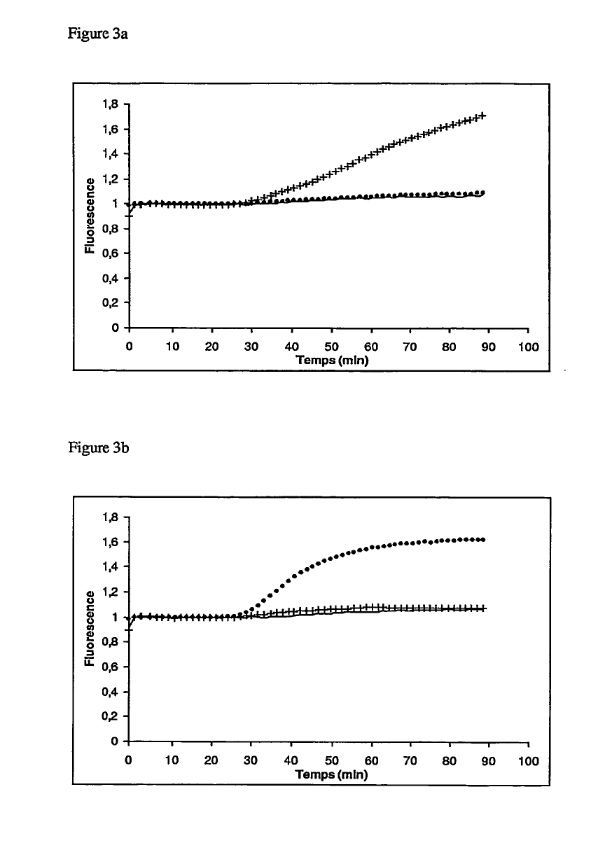

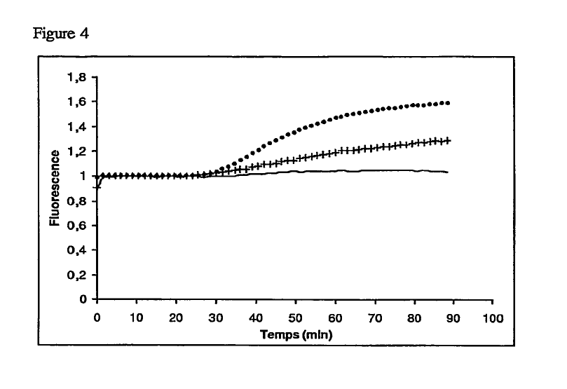

FIGS. 3 and 4 represent the genotyping of various cell lines for the factor II mutation +20210 G/A, by virtue of the simultaneous presence of the molecular beacons OGH 916 (SEQ ID No. 11, +20210-A) and OGH 1104 (SEQ ID No. 12, +2010-G) in the reaction mixture.

In this respect, FIG. 3a represents the genotyping of the GM14899 cell line homozygous for the factor II wild-type allele (+20210-G), while FIG. 3b represents the genotyping of the GM16000C cell line homozygous for the factor II mutated allele (+20210-A). The crosses represent the detection of the “molecular beacons” of SEQ ID No. 12, making it possible to reveal the wild-type allele, while the circles represent the detection of the “molecular beacons” of SEQ ID No. 11, allowing the detection of the factor II 20210 mutation.

FIG. 4 represents the genotyping of the GM16028B cell line heterozygous for the factor II +20210 mutation. The presence of the mutated allele (black circles) and of the wild-type allele (curved with crosses) is clearly detected. The crosses represent the detection of the “molecular beacons” of SEQ ID No. 12, making it possible to reveal the wild-type allele, while the circles represent the detection of the “molecular beacons” of SEQ ID No. 11, allowing the detection of the factor II 20210 mutation.

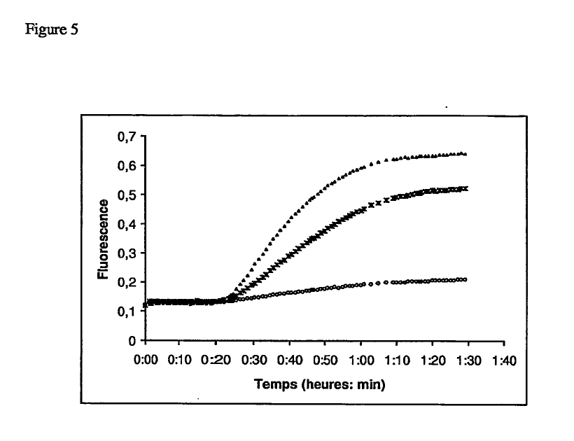

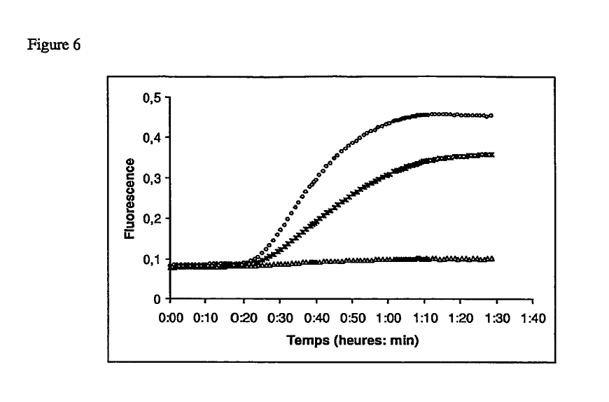

FIGS. 5 and 6 represent the genotyping of various cell lines (GM 14899: A/A; GM 16028B: A/G; GM 16000C: G/G) for the factor V +1691 mutation, by virtue of the presence of the molecular beacons SEQ ID Nos. 3 and 4 in the reaction mixture.

Thus, FIG. 5 represents the detection of the FAM beacon specific for the A allele of factor V (G+1691A) using DNA from the GM14899 line (A/A homozygous, mutated allele; triangles), GM16028B line (A/G heterozygous; crosses) and GM16000C line (GIG homozygous, wild-type allele; circles).

Thus, FIG. 6 represents the detection of the ROX beacon specific for the G allele of factor V (G+1691A) using DNA from the GM14899 line: A/A (homozygous, mutated allele; triangles); GM16028B line (A/G heterozygous; crosses) and GM 16000C line (G/G homozygous, wild-type allele, circles).

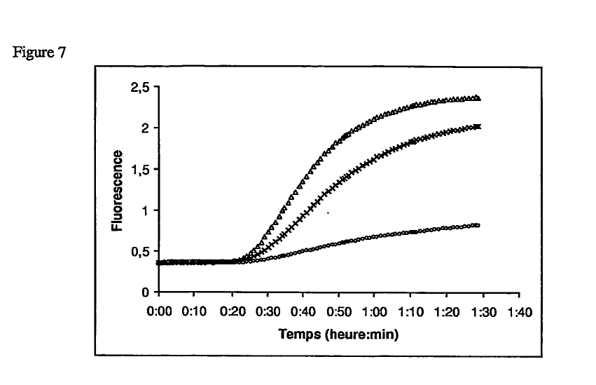

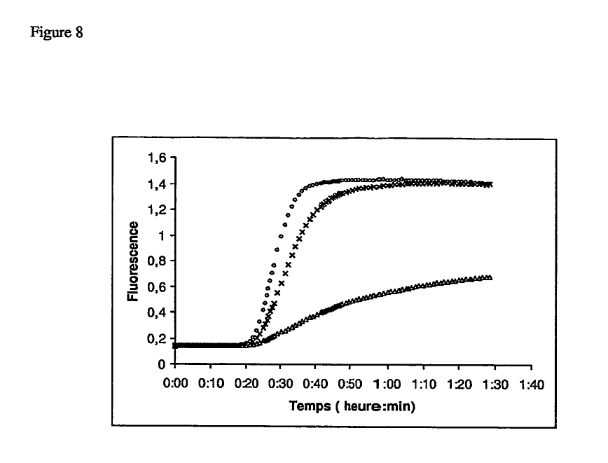

FIGS. 7 and 8 represent the genotyping of various cell lines (GM 14899: G/G; GM 16028B: A/G; GM 16000C: A/A) for the factor II +20210 mutation, by virtue of the presence of the molecular beacons SEQ ID Nos. 7 and 8 in the reaction mixture.

Thus, FIG. 7 represents the detection of the FAM beacon specific for the G allele of factor II (G+20210A) using DNA from the GM14899 line: G/G (homozygous, mutated allele; triangles); GM16028B line (A/G heterozygous; crosses) and GM 16000C line (A/A homozygous, wild-type allele, circles).

Thus, FIG. 8 represents the detection of the ROX beacons specific for the A allele of factor II (G+20210A) using DNA from the GM14899 line: G/G (homozygous, mutated allele; triangles); GM16028B line (A/G heterozygous; crosses) and GM 16000C line (A/A homozygous, wild-type allele, circles).

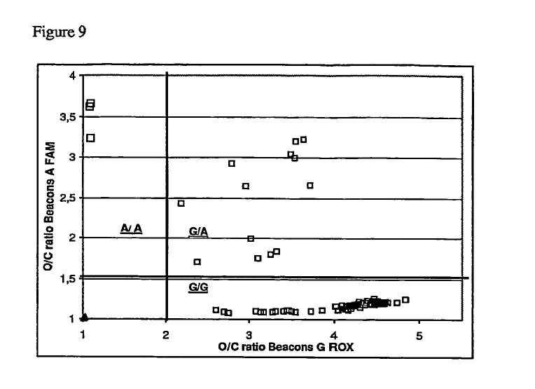

FIG. 9 is a graphic representation of 72 factor V samples (clinical and cell line samples). The samples are positioned according to their ratios of fluorescence intensity of the FAM (A allele-specific) and ROX (G allele-specific) beacons.

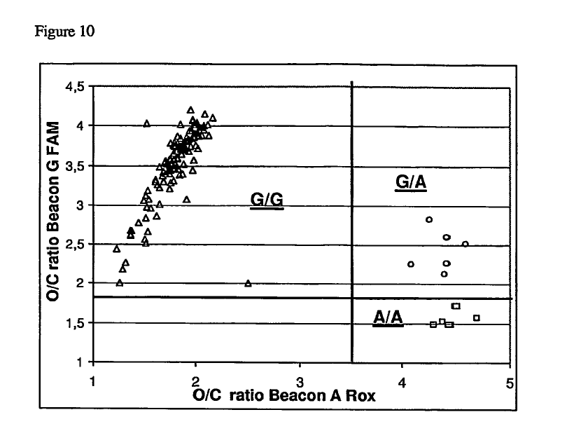

FIG. 10 is a graphic representation of 130 factor II samples (clinical and cell line samples). These samples are positioned according to their ratios of fluorescence intensity of the FAM (G allele-specific) and ROX (A allele-specific) beacons.

### EXAMPLE 1

1/ Sample and DNA Extraction

The method according to the invention was validated on cell lines, and also on clinical samples from patients.

Cell lines—Three lymphoblastoid cell lines of known genotype for the factor V Leiden mutations and the factor II 20210 mutation (Coriell cell repository) were used:

The GM14899 line expresses only the factor V Leiden mutation: this line is homozygous for the factor V Leiden mutation (A/A) and homozygous for the wild-type allele at position 20120 of factor II (G/G). The GM16000C line expresses only the factor II 20210 mutation: this line is homozygous for the factor II 20210 mutation (A/A) and homozygous for the wild-type allele at position 1691 of factor V (G/G). The GM16028B line is heterozygous for the 2 mutations (G/A).

These cell lines were placed in culture (37° C., 5% CO₂) in an RPMI 1640 medium supplemented with fetal bovine serum (15%), L-glutamine (2 mM), peni-streptomycin penicillin: 200 U/ml; streptomycin: 0.2 mg/ml).

The DNA extraction was carried out from a cell pellet of GM16000C, or from a cell pellet of GM16028B or of GM14899. The pelleted cells were subjected to hypotonic lysis and to enzymatic digestion with proteinase K. After deproteination, the DNA was precipitated with ethanol, dried, and dissolved in water. The quantity and quality of the DNA were determined by UV spectrophotometry.

Sample from patients—200 μl of blood taken from patients in which the presence of the Leiden mutation was known were used. The genomic DNA was extracted using the NucleoSpin kit (Marcherey-Nagel, Hoerdt, France), and taken up in 20 μl of DNAse-free water. The quantity and quality of the DNA were determined on an agarose gel containing ethidium bromide, and by UV spectrophotometry.

Cloning of the region of interest: In order to obtain a large amount of DNA of the region that may express the factor V Leiden mutation, a PCR was carried out using the genomic DNA as obtained above, from cell lines or blood samples, using a pair of amplification primers comprising SEQ ID No. 3, 5′ AGTGCTTAACAAGACCATACTA 3′ for the first primer, and SEQ ID No. 4, 5′ AACAGACCTGGAATTTGAAACTAA 3′ for the second primer. The parameters of the PCR were as follows: 2 min at 95° C.; 30 cycles of 30 s at 95° C., 30 s at 55° C., 30 s at 72° C.; followed by 7 min at 72° C. The amplicons thus obtained were cloned into a vector PCR-Trap (GeneHunter, USA) and verified by sequencing. The plasmids, containing either a nucleotide G or a nucleotide A at position 1691, were amplified and purified using a Plasmid Maxi kit (Qiagen; Germany).

In order to obtain a large amount of DNA of the region that may express the factor II 20210 mutation, a PCR was carried out using the genomic DNA as obtained above, from cell lines or from blood samples, using a pair of amplification primers comprising SEQ ID No. 7, 5′ TCTAGAAACAGTTGCCTGGC 3′ for the first primer, and SEQ ID No. 8, 5′ CTACCAGCGTGCCACCAGGT 3′ for the second primer. The parameters of the PCR were as follows: 2 min at 95° C.; 30 cycles of 30 s at 95° C., 30 s at 55° C., 30 s at 72° C.; followed by 7 min at 72° C. The amplicons thus obtained were cloned into a vector PCR-Trap (GeneHunter, USA) and verified by sequencing. The plasmids, containing either a nucleotide G or a nucleotide A at position 1691, were amplified and purified using a Plasmid Maxi kit (Qiagen; Germany).

2) Amplification by NASBA

In order to determine the genotyping of the DNA sample taken from a patient or from a cell line, an amplification reaction mixture (40 mM Tris HCl, pH 8.5; 12 mM MgCl₂; 70 mM KCl; 5 mM dithiothreitol; 15% v/v DMSO; 1 mM dNTP) containing the amplification primers for amplifying either the region that may contain the Leiden mutation, or the region that may contain the factor II 20210 mutation, and detection probes specific for each mutation was prepared.

Thus, the reaction medium for detecting the presence of the factor V Leiden mutation comprised:
0.2 μM (final concentration) of a first amplification primer of SEQ ID No. 2, 5′ AGT GCT TAA CAA GAC CAT ACT A 3′,
0.2 μM (final concentration) of a second amplification primer of SEQ ID No. 1, 5′ AAA TTC TCA GAA TTT CTG AAA GG 3′ comprising the T7 phage polymerase promoter, i.e. an amplification primer whose complete sequence corresponds to SEQ ID No. 13, 5′ aat tct aat acg act cac tat agg gag aAA ATT CTC AGA ATT TCT GAA AGG 3′,
0.2 μM (final concentration) of “molecular beacons” of SEQ ID No. 9, 5′ CTG GAC AGG CGA IGA A 3′, labeled with a ROX (6-carboxy-X-rhodamine) fluorophore in the 5′ position, and of a “quencher” (Dabsyl) in the 3′ position (complete sequence: 5′ ROX-cgatcg CTGGACAGGCGAIGAAcgatcg-Dabsyl 3′). This “molecular beacon” made it possible to reveal the absence of the Leiden mutation,
0.1 μM (final concentration) of “molecular beacons” of SEQ ID No. 10, 3′ CTG GAC AGG CAA IGA A 3′, labeled with a FAM (6-carboxyfluorescein) fluorophore in the 5′ position, and of a “quencher” (Dabsyl) in the 3′ position (complete sequence: 5′ FAM-cgatcg CTGGACAGGCAAIGAAcgatcg-Dabsyl 3′). This “molecular beacon” made it possible to reveal the presence of the Leiden mutation.

For factor II, the following were added to the reaction medium:
0.2 μM (final concentration) of a first amplification primer of SEQ ID No. 6, 5′ TTC TGG GCT CCT GGA ACC AA 3′
0.2 μM (final concentration) of a second amplification primer of SEQ ID No. 5, 5′ ATT ACT GGC TCT TCC TGA GC 3′, comprising the T7 phage polymerase promoter, i.e. an amplification primer whose complete sequence corresponds to SEQ ID No. 14,
0.1 μM (final concentration) of “molecular beacons” of SEQ ID No. 11, 5′ ACT CTC AGC AAG CCT CAA 3′, labeled with a ROX (6-carboxy-X-rhodamine) fluorophore in the 5′ position and of a “quencher” (Dabsyl) in the 3′ position (complete sequence: 5′ ROX-cga tcg ACT CTC AGC AAG CCT CAA cga tcg-Dabsyl 3′). This “molecular beacon” made it possible to reveal the presence of the factor II 20210 mutation,
0.2 μM (final concentration) of “molecular beacons” of SEQ ID No. 12, 5′ ACT CTC AGC GAG ICT CAA 3′, labeled with a FAM (6-carboxyfluorescein) fluorophore in the 5′ position, and of a “quencher” (Dabsyl) in the 3′ position (complete sequence: 5′ FAM-cgg tcg ACT CTC AGC GAG ICT CAA cga ccg-Dabsyl 3′). This “molecular beacon” made it possible to reveal the absence of the factor II 20210 mutation.

As shown in FIG. 1, the “molecular beacon” of SEQ ID No. 9, labeled with a ROX (6-carboxy-X-rhodamine) fluorophore is very specific for the wild-type allele: in fact, the fluorescence was exponential, before the appearance of a plateau, when the reaction was carried out using a cell line not expressing the Leiden mutation (curve of white squares; FIG. 1a), whereas the fluorescence remained baseline when the reaction was carried out using a cell line expressing the Leiden mutation (curve of white squares: FIG. 1b). The “molecular beacons” of SEQ ID No. 10, labeled with a FAM (6-carboxyfluorescein) fluorophore is specific with the mutated allele. In fact, the fluorescence was exponential when the reaction was carried out using a cell line expressing the Leiden mutation (curve of triangles; FIG. 1b), while the fluorescence remained baseline when the reaction was carried out using a cell line not expressing the Leiden mutation (curve of triangles; FIG. 1a).

By using 2 different fluorophores, the “molecular beacon” of SEQ ID No. 9 could be used simultaneously with a “molecular beacon” of SEQ ID No. 10, which made it possible to very rapidly detect, using a single reaction, the presence or absence of the factor V Leiden mutation. The results are given in FIG. 2, obtained from a cell line heterozygous for the factor V Leiden mutation.

These two “molecular beacons”, used simultaneously with the amplification primers of SEQ ID No. 1 and SEQ ID No. 2 therefore make it possible to differentiate clearly between the two alleles.

Comparable results were obtained using clinical samples.

The results obtained can also be expressed in terms of the ratio between the fluorescence emitted when the plateau is reached and the initial fluorescence (OC ratios (open/close ratios)): the higher the ratio, the more specific the hybridization between molecular beacon and amplicon and the greater the discrimination between the two alleles.

The results are given in Table 1 below:

TABLE 1
O/C ratios obtained on cell lines and blood samples expressing or not
expressing the Leiden mutation

| | Line GM | Line GM | Line GM | Clinical | Clinical | Clinical |
|---|---|---|---|---|---|---|
| | 1600C | 16028B | 14899 | sample | sample | sample |
| | Factor V | Factor V | Factor V | Factor V | Factor V | Factor V |
| O/C RATIO | G/G | G/A | A/A | G/A | G/G | A/A |
| Beacons of | 1.33 | 2.57 | 2.83 | 1.96 | 1.41 | 3.03 |
| SEQ ID No. 10 | | | | | | |
| Beacons of | 3.77 | 3.04 | 1.13 | 3.05 | 3.74 | 1.14 |
| SEQ ID No. 9 | | | | | | |

These results demonstrate that the use of a “molecular beacon” of SEQ ID No. 9 simultaneously with the NASBA amplification with a pair of amplification primers, comprising a first primer comprising SEQ ID No. 1 and a second primer comprising SEQ ID No. 2, made it possible to detect very specifically the absence of the Leiden mutation. The use of a “molecular beacon” of SEQ ID No. 10 simultaneously with NASBA amplification with a pair of amplification primers, comprising a first primer comprising SEQ ID No. 1 and a second primer comprising SEQ ID No. 2, made it possible to detect very specifically the presence of the Leiden mutation.

As shown in FIG. 3, the “molecular beacon” of SEQ ID No. 12, labeled with a FAM (6-carboxyfluorescein) fluorophore is very specific for the factor II wild-type allele. In fact, the fluorescence was exponential, when the reaction was carried out using a cell line not expressing the 20210 mutation (curve of crosses; FIG. 3a), while the fluorescence remained baseline when the reaction was carried out using a cell line expressing this mutation (curve of crosses; FIG. 3b).

The “molecular beacons” of SEQ ID No. 11, labeled with a ROX (6-carboxy-X-rhodamine) fluorophore is specific for the mutated allele. The fluorescence was exponential, when the reaction was carried out using a cell line expressing the 20210 mutation (curve of circles; FIG. 3b), while the fluorescence remained baseline when the reaction was carried out using a cell line not expressing this mutation (curve of circles; FIG. 3a).

By using 2 different fluorophores, the “molecular beacon” of SEQ ID No. 11 could be used simultaneously with a “molecular beacon” of SEQ ID No. 12, which made it possible to very rapidly detect, using a single reaction, the presence or absence of the factor II 20210 mutation. This is what is represented in FIG. 4, obtained from a cell line homozygous for the factor II 20210 mutation. Comparable results are obtained using a clinical example.

Furthermore, according to a comparable protocol, 203 clinical DNAs were genotyped. All the results obtained were compared with the results obtained by means of a standard technique, RFLP. This study showed 100% agreement between the 2 genotyping methods.

With the aim of identifying the presence or absence of the various factor II and factor V alleles in the same tube, assays consisting of simultaneous amplification of the two genes in the presence of 4 “molecular beacons” specific for each allele were carried out. The same “molecular beacons” and pairs of amplification primers as for the amplifications of the genes alone were used, at the same concentrations.

### EXAMPLE 2

1/ Sample and DNA Extraction

The method according to the invention was validated on cell lines and also on clinical samples from patients.

Cell lines—Three lymphoblast cell lines of known genotype for the factor V Leiden mutations and the factor II 20210 mutation (Coriell cell repository) were used:

The GM14899 line expresses only the factor V Leiden mutation: this line is homozygous for the factor V Leiden mutation (A/A) and homozygous for the wild-type allele at position 20120 of factor II (G/G). The GM16000C line expresses only the factor II 20210 mutation: this line is homozygous for the factor II 20210 mutation (A/A) and homozygous for the wild-type allele at position 1691 of factor V (G/G). The GM16028B line is heterozygous for the 2 mutations (G/A).

These cell lines were placed in culture (37° C., 5% CO₂) in an RPMI 1640 medium supplemented with fetal bovine serum (15%), L glutamine (2 mM), peni-streptomycin (penicillin: 200 U/ml; streptomycin: 0.2 mg/ml).

The DNA extraction was carried out from a cell pellet of GM16000C, or from a cell pellet of GM16028B or of GM14899. The pelleted cells were subjected to hypotonic lysis and to enzymatic digestion with proteinase K. After deproteination, the DNA was precipitated with ethanol, dried, and dissolved in water. The quantity and quality of the DNA was determined by UV spectrophotometry.

Sample from patients—100 μl of blood taken from ETS (Etablissement Français du sang [French Blood Bank]; Lyons) normal donors for whom the FII/FV typing is unknown but tested in parallel using a standard LightCycler technique. The genomic DNA was extracted using the NucliSens Magnetic Extraction reagent and NucliSens Lysis Buffer kit and the miniMag instrument (cf. miniMag bulletin except Wash3 washing (10 sec instead of 15 sec) and the DNA was eluted in 80 μl of elution buffer (10 min at 70° C.+mix at 14 000 rpm).

The quantity and quality of the DNA were determined by UV spectrophotometry.

Cloning of the region of interest: In order to obtain a large amount of DNA of the region that may express the factor V Leiden mutation, a PCR was carried out using the genomic DNA as obtained above, from cell lines or from blood samples, using a pair of amplification primers comprising SEQ ID No. 3, 5′ AGTGCTTAACAAGACCATACTA 3′ for the first primer, and SEQ ID No. 4, 5′ AACAGACCTGGAATTTGAAACTAA 3′ for the second primer. The parameters of the PCR were as follows: 2 min at 95° C.; 30 cycles of 30 s at 95° C., 30 s at 55° C., 30 s at 72° C.; followed by 7 min at 72° C.). The amplicons thus obtained were cloned into a vector PUC19 and verified by sequencing. The plasmids, containing either a nucleotide G or a nucleotide A at position 1691, were amplified and purified using a plasmid Maxi kit (Qiagen; Germany).

In order to obtain a large amount of DNA of the region that may express the factor II 20210 mutation, a PCR was carried out using the genomic DNA as obtained above, from cell lines or from blood samples, using a pair of amplification primers comprising SEQ ID No. 7, 5′ TCTAGAAACAGTTGCCTGGC 3′ for the first primer, and SEQ ID No. 8, 5′ CTACCAGCGTGCCACCAGGT 3′ for the second primer. The parameters of the PCR were as follows: 2 min at 95° C.; 30 cycles of 30 s at 95° C., 30 s at 55° C., 30 s at 72° C.; followed by 7 min at 72° C. The amplicons thus obtained were cloned into a vector PCR-Trap (GeneHunter, USA) and verified by sequencing. The plasmids, containing either a nucleotide G or a nucleotide A at position 1691, were amplified and purified using a plasmid Maxi kit (Qiagen; Germany).

2) Amplification by NASBA

In order to determine the genotyping of the DNA taken from a patient or from a cell line, a factor V amplification reaction mixture (40 mM Tris HCl, pH 8.5; 12 mM MgCl₂; 100 mM KCl; 5 mM dithiothreitol; 15% v/v DMSO; 1 mM dNTP) containing the amplification primers for amplifying the region that may contain the FV Leiden mutation:
0.2 μM (final concentration) of a first amplification primer of SEQ ID No. 2, 5′ AGT GCT TAA CAA GAC CAT ACT A 3′,
0.2 μM (final concentration) of a second amplification primer of SEQ ID No. 1, 5′ AAA TTC TCA GAA TTT CTG AAA GG 3′ comprising the T7 phage polymerase promoter, i.e. an amplification primer whose complete sequence corresponds to SEQ ID No. 13, 5′ aat tct aat acg act cac tat agg gag aAA ATT CTC AGA ATT TCT GAA AGG 3′,
0.05 μM (final concentration) of “molecular beacons” of SEQ ID No. 9, 5′ CTG GAC AGG CGA IGA A 3′, labeled with a ROX (6-carboxy-X-rhodamine) fluorophore in the 5′ position, and of a “quencher” (Dabsyl) in the 3′ position (complete sequence: 5′ ROX-cgatcg CTGGACAGGCGAIGAAcgatcg-Dabsyl 3′). This “molecular beacon” made it possible to reveal the absence of the Leiden mutation,
0.025 μM (final concentration) of “molecular beacons” of SEQ ID No. 10, 3′ CTG GAC AGG CAA IGA A 3′, labeled with a FAM (6-carboxy-fluorescein) fluorophore in the 5′ position, and of a “quencher” (Dabsyl) in the 3′ position (complete sequence: 5′ FAM-cgatcg CTGGACAGGCAAIGAAcgatcg-Dabsyl 3′). This “molecular beacon” made it possible to reveal the presence of the Leiden mutation.

In order to determine the genotyping of the DNA taken from a patient or from a cell line, a factor II amplification reaction mixture (40 mM Tris HCl, pH 8.5; 12 mM MgCl₂; 70 mM KCl; 5 mM dithiothreitol; 15% v/v DMSO; 1 mM dNTP) containing the amplification primers for amplifying the region that may contain the FII +20210 mutation:

For factor II, the following were added to the reaction medium:
0.2 μM (final concentration) of a first amplification primer of SEQ ID No. 16, 5′ CTGGAACCAATCCCGTGAAAG 3′,
0.2 μM (final concentration) of a second amplification primer of SEQ ID No. 15, 5′ AGCTGCCCATGAATAGCACT 3′, also comprising the T7 phage polymerase promoter, i.e. corresponding to SEQ ID No. 19, aattctaatacgactcactataggAGCTGCCCATGAATAGCACT,
0.1 μM (final concentration) of “molecular beacons” of SEQ ID No. 17, 5′ ACT CTC AGC AAG CCT CAA 3′, labeled with a ROX (6-carboxy-X-rhodamine) fluorophore in the 5′ position, and of a “quencher” (Dabsyl) in the 3′ position (complete sequence: 5′ ROX-cga tog ACT CTC AGC AAG CCT CAA cga tcg-Dabsyl 3′). This “molecular beacon” made it possible to reveal the presence of the factor II 20210 mutation,
0.1 μM (final concentration) of “molecular beacons” of SEQ ID No. 18, 5′ TCTCAGCGGGCCTCA 3′, labeled with a FAM (6-carboxyfluorescein) fluorophore in the 5′ position, and of a “quencher” (Dabsyl) in the 3′ position (complete sequence: 5′ FAM-cgtcg TCTCAGCGGGCCTCA cgacg-Dabsyl 3′). This “molecular beacon” made it possible to reveal the absence of the factor II 20210 mutation.

As shown in FIG. 5, the “molecular beacon” of SEQ No. 9 labeled with a FAM fluorophore is very specific for the mutated allele: in fact, the fluorescence was exponential, before the appearance of a plateau, when the reaction was carried out using cell lines expressing the Leiden mutation (curves of triangles; and of crosses, FIG. 5), while the fluorescence remained baseline when the reaction was carried out using a cell line not expressing the Leiden mutation (white circle curve; FIG. 5).

The “molecular beacon” of SEQ ID No. 10, labeled with a ROX fluorophore, is specific for the wild-type allele. In fact, the fluorescence was exponential when the reaction was carried out using cell lines expressing the Leiden wild-type genotype (curve of circles and crosses; FIG. 6), while the fluorescence remained baseline when the reaction was carried out using a cell line not expressing the wild-type genotype (FIG. 6, curve of triangles).

By using 2 different fluorophores, the “molecular beacon” of SEQ ID No. 10 is used simultaneously with a “molecular beacon” of SEQ ID No. 9, which made it possible to very rapidly detect, using a single reaction, the presence and/or absence of the mutation for the two factor V gene alleles. By virtue of the analysis of FIGS. 5 and 6, it is therefore possible to determine whether the sample has the G/G, G/A, or A/A genotype. These two “molecular beacons”, used simultaneously with the amplification primers of SEQ ID No. 2 and SEQ ID No. 1, therefore make it possible to differentiate clearly between the two alleles.

Comparable results are obtained using samples of fresh blood taken from l'Etablissement de Transfusion Francis (ETS) [French Blood Bank] donors.

The results obtained can also be expressed in terms of the ratio between the fluorescence emitted when the plateau is reached and the initial fluorescence (O/C ratios (open/close ratios)): the higher the ratio, the more specific the hybridization between molecular beacon and amplicon and the greater the discrimination between the two alleles.

By virtue of the cell lines, positivity detection limits (threshold or cutoff) were established for the FAM and ROX beacons.

The results of 72 cell line samples are represented in FIG. 9.

Each “square” symbol represents a sample tested and positioned according to the value of the O/C ratio of the FAM beacons and the OC ratio of the ROX beacon for this sample. By virtue of the positivity detection limits for these two beacons, it is thus possible to determine graphically the factor V Leiden typing for 72 samples.

A sample is classified A/A when its mutated allele (A)-specific FAM beacon O/C ratio is greater than 1.5 and when its wild-type allele (G)-specific ROX beacon O/C ratio is less than 2.

A sample is classified G/G when its mutated allele (A)-specific FAM beacon O/C ratio is less than 1.5 and when its wild-type allele (G)-specific ROX beacon O/C ratio is greater than 2.

A sample is classified G/A when its mutated allele (A)-specific FAM beacon O/C ratio is greater than 1.5 and when its wild-type allele (G)-specific ROX beacon O/C ratio is greater than 2.

These results demonstrate that the use of a “molecular beacon” of SEQ ID No. 9 simultaneously with NASBA amplification with a pair of amplification primers, comprising a first primer comprising SEQ ID No. 1 and a second primer comprising SEQ ID No. 2, made it possible to detect very specifically the absence of the Leiden mutation. The use of a “molecular beacon” of SEQ ID No. 10 simultaneously with NASBA amplification with a pair of amplification primers, comprising a first primer comprising SEQ ID No. 1 and a second primer comprising SEQ ID No. 2, made it possible to detect very specifically the presence of the Leiden mutation.

As shown in FIG. 7, the “molecular beacon” of SEQ No. 18, labeled with a FAM fluorophore, is very specific for the wild-type allele: in fact, the fluorescence was exponential, before the appearance of a plateau, when the reaction was carried out using cell lines expressing the factor II wild-type (curves of triangles; and of crosses, FIG. 7), while the fluorescence remained baseline when the reaction was carried out using a cell line not expressing the factor II wild-type polymorphism (circle curve; FIG. 7).

The “molecular beacon” of SEQ ID No. 17, labeled with a ROX fluorophore, is specific for the mutated allele. In fact, the fluorescence was exponential when the reaction was carried out using cell lines expressing the factor II mutation (curve of circles and crosses; FIG. 6), while the fluorescence remained baseline when the reaction was carried out using a cell line not expressing the wild-type genotype (FIG. 6, curve of triangles).

By using 2 different fluorophores, the “molecular beacon” of SEQ ID No. 17 is used simultaneously with a “molecular beacon” of SEQ ID No. 18, which made it possible to very rapidly detect, using a single reaction, the presence or absence of the mutation for the factor II gene. By virtue of the analysis of FIGS. 7 and 8, it is therefore possible to determine whether the sample has the G/G, G/A or A/A genotype.

The results of 130 samples comprising cell lines (squares) and samples extracted from fresh blood (triangles and circles) are represented in FIG. 10.

Each symbol represents a sample tested and positioned according to the value of the O/C ratio of the FAM beacon and the O/C ratio of the ROX beacon for this sample. By virtue of the positivity detection limits of these two beacons, it is thus possible to determine graphically the factor II typing for 130 samples.

A sample is classified A/A when its mutated allele (A)-specific ROX beacon O/C ratio is greater than 3.5 and when its wild-type allele (G)-specific FAM beacon O/C ratio is less than 1.8.

A sample is classified G/G when its mutated allele (A)-specific ROX beacon O/C ratio is less than 3.5 and when its wild-type allele (G)-specific FAM beacon O/C ratio is greater than 1.8.

A sample is classified G/A when its mutated allele (A)-specific ROX beacon O/C ratio is greater than 3.5 and when its wild-type allele (G)-specific FAM beacon O/C ratio is greater than 1.8.

Furthermore, according to a comparable protocol, the samples of fresh blood were genotyped. All the results obtained were compared with the results obtained using a standard RT-PCR technique (Lightcycler, Roche). This study showed 100% agreement between the 2 genotyping methods.

With the aim of identifying the presence or absence of the various factor II and factor V alleles in the same tube, assays consisting of simultaneous amplification of the two genes in the presence of 4 “molecular beacons” specific for each allele were carried out. The same “molecular beacons” and pairs of amplification primers as for the amplification of the genes alone were used, at the same concentrations.

1. An in vitro method for the diagnosis/prognosis of thrombosis, comprising:
A. extracting nucleic acid material from a biological sample;
B. obtaining amplicons of at least one target sequence of the nucleic acid material using at least one pair of amplification primers; and
C. detecting the presence of said amplicons using at least one detection probe;
characterized in that, in (B), said pair of primers comprises at least one amplification primer comprising at least 10 nucleotide units of a nucleotide sequence chosen from SEQ ID NO: 1, 2, 3, 4, 5, 6, 7, 8, 15 or 16.

2. The method as claimed in claim 1, characterized in that, in (C), said detection probe comprises at least 10 nucleotide units of a nucleotide sequence chosen from SEQ ID NO: 9, 10, 11, 12, 17, or 18.

3. The method as claimed in claim 1, characterized in that, in (B), said pair of primers is chosen from the following pairs of primers:
a first amplification primer comprising at least 10 nucleotide units of the nucleotide sequence SEQ ID NO: 1 and a second amplification primer comprising at least 10 nucleotide units of the nucleotide sequence SEQ ID NO: 2;
a first amplification primer comprising at least 10 nucleotide units of the nucleotide sequence SEQ ID NO: 3 and a second amplification primer comprising at least 10 nucleotide units of the nucleotide sequence SEQ ID NO: 4;
a first amplification primer comprising at least 10 nucleotide units of the nucleotide sequence SEQ ID NO: 5 and a second amplification primer comprising at least 10 nucleotide units of the nucleotide sequence SEQ ID NO: 6;
a first amplification primer comprising at least 10 nucleotide units of the nucleotide sequence SEQ ID NO: 7 and a second amplification primer comprising at least 10 nucleotide units of the nucleotide sequence SEQ ID NO: 8; or
a first amplification primer comprising at least 10 nucleotide units of the nucleotide sequence SEQ ID NO: 15 and a second amplification primer comprising at least 10 nucleotide units of the nucleotide sequence SEQ ID NO: 16.

4. The method as claimed in claim 1, in which said pair of primers comprises at least one amplification primer comprising a promoter allowing the initiation of transcription by a T7 bacteriophage polymerase.

5. The method as claimed in claim 1, in which, in (C), the detection probe comprises a fluorophore and a quencher.

6. An amplification primer comprising at least 10 nucleotide units of a nucleotide sequence chosen from SEQ ID NO: 1, 2, 3, 4, 5, 6, 7, 8, 15 and 16.

7. The amplification primer as claimed in claim 6, comprising a promoter allowing the initiation of transcription by a T7 bacteriophage polymerase.

8. A pair of amplification primers chosen from the following pairs of primers:
a first amplification primer comprising at least 10 nucleotide units of the nucleotide sequence SEQ ID NO: 1 and a second amplification primer comprising at least 10 nucleotide units of the nucleotide sequence SEQ ID NO: 2;
a first amplification primer comprising at least 10 nucleotide units of the nucleotide sequence SEQ ID NO: 3 and a second amplification primer comprising at least 10 nucleotide units of the nucleotide sequence SEQ ID NO: 4;
a first amplification primer comprising at least 10 nucleotide units of the nucleotide sequence SEQ ID NO: 5 and a second amplification primer comprising at least 10 nucleotide units of the nucleotide sequence SEQ ID NO: 6;
a first amplification primer comprising at least 10 nucleotide units of the nucleotide sequence SEQ ID NO: 7 and a second amplification primer comprising at least 10 nucleotide units of the nucleotide sequence SEQ ID NO: 8; or
a first amplification primer comprising at least 10 nucleotide units of the nucleotide sequence SEQ ID NO: 15 and a second amplification primer comprising at least 10 nucleotide units of the nucleotide sequence SEQ ID NO: 16.

9. The pair of primers as claimed in claim 8, in which said first primer comprises a promoter allowing the initiation of transcription by a T7 bacteriophage polymerase.

10. An amplification method comprising including at least one amplification primer as claimed in claim 6 in a NASBA amplification reaction.

11. A method for the diagnosis/prognosis of thrombosis, comprising using at least one primer as claimed in claim 6 as a reagent for the diagnosis/prognosis of thrombosis.

12. A kit for the diagnosis/prognosis of thrombosis, comprising at least one primer as claimed in claim 6.

13. An amplification method comprising including at least one pair of primers as claimed in claim 8 in a NASBA amplification reaction.

14. A method for the diagnosis/prognosis of thrombosis, comprising using at least one pair of primers as claimed in claim 8 as a reagent for the diagnosis/prognosis of thrombosis.

15. A kit for the diagnosis/prognosis of thrombosis, comprising at least one pair of primers as claimed in claim 8.
